(19) United States
(12) Patent Application Publication
(10) Pub. No.: US 2024/0390186 A1
(43) Pub. Date: Nov. 28, 2024

(54) FORKED PHACOEMULSIFICATION NEEDLE

(71) Applicant: Johnson & Johnson Surgical Vision, Inc., Irvine, CA (US)

(72) Inventor: Chetan Nirkhe, Aliso Viejo, CA (US)

(21) Appl. No.: 18/324,916

(22) Filed: May 26, 2023

(51) Int. Cl. A61F 9/007; A61B 17/32; A61M 1/00

(52) U.S. Cl. CPC A61F 9/00745; A61B 17/320068; A61M 1/00; A61B 2017/320098; A61M 2210/0612

(57) ABSTRACT

A needle for use with a phacoemulsification handpiece is disclosed. The needle may compromise an elongated needle body having a proximal end and a distal end terminating at a junction point; a first tine having a proximal end and a distal end terminating in a first tip; a second tine having a proximal end and a distal end terminating in a second tip, wherein the proximal end of the first tine and the proximal end of the second tine are each connected at the junction point.

### FIELD OF INVENTION

This present invention relates generally to the field of ocular surgery and more particularly to a needle for a phacoemulsification surgical instrument.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this a physician may recommend phacoemulsification cataract surgery. Before the procedure the surgeon numbs the area with anesthesia. Then a small incision is made in the cornea or sclera of the eye. Fluids, such as viscoelastic, may be injected into this incision to protect and support the surrounding structures. The anterior surface of the lens capsule is then removed to gain access to the cataractic lens. The surgeon then uses a phacoemulsification handpiece to remove the lens. The handpiece having at the distal end a needle that vibrates at ultrasonic frequency to sculpt/emulsify the lens and a sleeve substantially surrounding the needle. The needle has an aspiration port at the distal tip and the sleeve has one or more irrigation ports. One or more pumps (e.g., peristaltic, Venturi, progressive cavity, etc.) are fluidly coupled with the handpiece via the needle and an aspiration line to aspirate lens material and an irrigation fluid source (e.g., balanced salt solution) is fluidly coupled with the handpiece via the sleeve and an irrigation line. The one or more pumps are typically controlled with a microprocessor. The intraocular pressure within the eye is maintained by balancing the fluids and material aspirated from the eye and the irrigation fluid (e.g., balanced salt solution) administered to the eye. After removing the cataract with phacoemulsification, the softer outer lens cortex is removed with suction. An intraocular lens (IOL) having one or more haptics is introduced into the empty lens capsule. Once correctly implanted the IOL restores the patient's vision.

A phacoemulsification needle for more efficient emulsification of the lens is disclosed.

### SUMMARY

A needle for use with a phacoemulsification handpiece is disclosed. The needle may compromise an elongated needle body having a proximal end and a distal end terminating at a junction point; a first tine having a proximal end and a distal end terminating in a first tip; a second tine having a proximal end and a distal end terminating in a second tip, wherein the proximal end of the first tine and the proximal end of the second tine are each connected at the junction point. The first tine and the second tine may each have an outward curve to form a U-shape. The distance between the first tip and the second tip may be larger than the distance between the proximal end of the first tine and the proximal end of the second tine near the junction point to form a V-shape. The first tip and second tip may be straight or beveled. The first tine, and the second tine may include an aspiration lumen therethrough. The needle body may include internal threads. The needle may include one or more additional tines. The additional tines may or may not be in the same plane as the first tine and the second tine.

Also disclosed is a tool for installing a needle in a phacoemulsification handpiece. The tool may include a first vertical portion and a second vertical portion, wherein the first vertical portion includes a first hollow portion and the second vertical portion includes a second hollow portion; and a first horizontal portion connecting the first vertical portion and the second vertical portion, wherein the first hollow portion and second hollow portion each have a circumference substantially similar to a first tine and a second tine of the phacoemulsification needle and are configured to receive the first tine and the second tine.

Other systems, methods, features and advantages of the invention will be or will become apparent to one of skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The organization and manner of the structure and function of the disclosure, together with the further objects and advantages thereof, may be understood by reference to the following description taken in connection with the accompanying drawings, and in which:

FIG. 1A is a front view of a “U-shaped” phacoemulsification needle, according to an example of the present disclosure;

FIG. 1B is a cross sectional view of a portion of FIG. 1A;

FIG. 1C is a front view of a “U-shaped” phacoemulsification needle, according to an example of the present disclosure;

FIG. 2 is front view of a phacoemulsification needle with two straight tips, according to an example of the present disclosure;

FIG. 3 is front view of a phacoemulsification needle with one straight tip and one beveled tip, according to an example of the present disclosure;

FIG. 4 is front view of a phacoemulsification needle with two beveled tips, according to an example of the present disclosure;

FIG. 5 is a front view of a “V-shaped” phacoemulsification needle, according to an example of the present disclosure;

FIG. 6 is a front view of a phacoemulsification needle with three tips, according to an example of the present disclosure;

FIG. 7a is a cross-sectional view of a phacoemulsification needle assembly tool, according to an example of the present disclosure;

FIG. 7b is a front view of a phacoemulsification needle assembly tool, according to an example of the present disclosure;

FIG. 8a is a cross-sectional view of a phacoemulsification needle assembly tool, according to an example of the present disclosure;

FIG. 8b is a front view of a phacoemulsification needle assembly tool, according to an example of the present disclosure; and

FIG. 9 is a front view of a phacoemulsification needle assembly tool, according to an example of the present disclosure.

### DETAILED DESCRIPTION OF THE EXAMPLES

A phacoemulsification needle for more efficient emulsification of the lens is disclosed. The distal end of the disclosed phacoemulsification needle has a U-shape. The U-shaped distal end may include two tines that enable the needle to dig into the lens material and be more efficient in the sculpt and quadrant technique to break up the lens during phacoemulsification with or without ultrasonic power application. With the characteristic “U” shape defined by the needle geometry, the resonance of the needle is improved with respect to the material of construction of the needle and the frequency for ultrasonic vibration used, much akin to the tuning fork phenomenon. The result of which is expected to be a more uniform motion and consistent amplitude in the longitudinal and transverse direction due to symmetrical distribution of weight of material around the central or longitudinal axis. With longitudinal movement, the needle moves forward and backwards along the longitudinal axis of the handpiece, and the movement of the tines is also expected to move back and forth along the longitudinal axis of the handpiece with equal amplitudes resulting in cuts in the lens tissue. Such a tip motion may be desired for efficient emulsification of the lens since now you have two sharp metal features cutting lens in two different sections of the lens at the same time. Thus, a better sculpting can be achieved by the surgeon with multiple cuts on the lens without having to move the tip into different planes. The tines may also move in a transverse motion, which is a side to side motion perpendicular to the longitudinal axis of the handpiece, and such aside to side motion will result in the tines moving more or less in the same plane. In an example, the tines may move in a combination of longitudinal and transverse motion resulting in an elliptical movement of the tines as seen with an ELLIPS FX Handpiece. With the present invention, one would observe two elliptical motions occurring in parallel at the same time for the two tines, which would provide emulsification of a wider section of the lens resulting in better efficiency. The phacoemulsification needles described herein may be comprised of titanium, or any other material used for phacoemulsification tips. Also disclosed is a tool to assemble and disassemble the phacoemulsification needle.

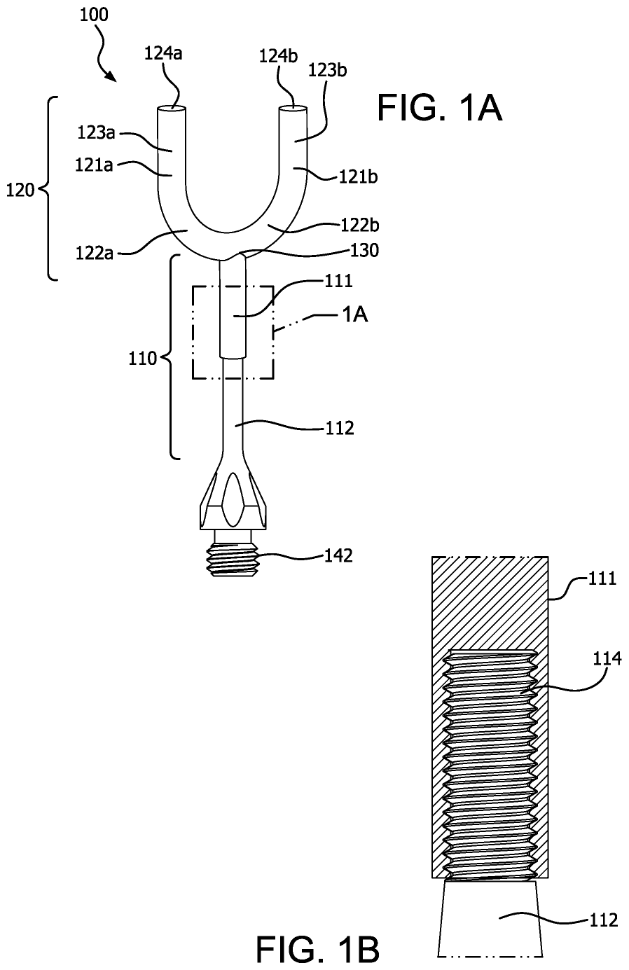

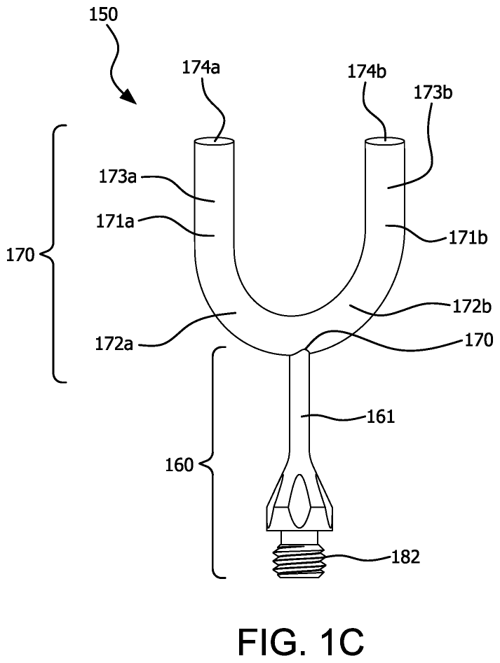

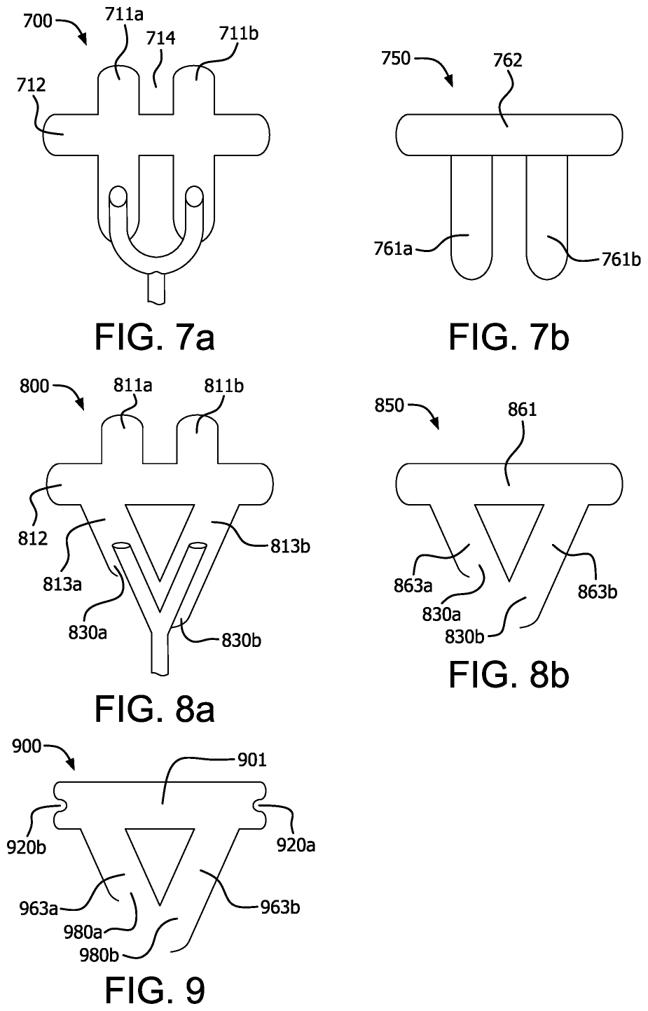

FIG. 1A is a view of a needle 100 of a phacoemulsification handpiece, according to an example. The needle 100 may include a proximal end 110 and a distal end 120. The proximal end 110 may include an elongated needle body 111 and bottom portion 112. The bottom portion 112 of the needle 100 is the standard threaded part of a phacoemulsification needle available on previous designs of needles offered in the market. This bottom portion 112 is configured to mate with a distal end of a handpiece. The bottom portion 112 may be screwed on to the distal end of the handpiece via the threads 142. The bottom portion 112 may be screwed on the distal end of the handpiece using the tool shown in FIGS. 7-9 that couples with at least the tines of the needle 100. The tines 121a and 121b and needle body 111 may be hollow to facilitate aspiration of the lens material and fluid during phacoemulsification.

The elongated needle body 111 may include an inner surface and a central channel or lumen coupled with one or more pumps to aspirate the emulsified lens particles from the eye. As shown in FIG. 1B, the inner surface of the elongated needle body 111 may also include internal threads that mate with external threads 114 of the bottom portion 112.

In some examples, the distal end 120 of the needle 100 may be a U-shape and may include a first tine 121a and a second tine 121b. The first tine 121a may include a proximal end 122a and a distal end 123a that terminates in a tip 124a. The second tine 121b may include a proximal end 122b and a distal end 123b that terminates in a tip 124b. The first tine 121a and second tine 121b may be connected to the elongated body 111 at a junction point 130. The U-shaped distal end 120 may help dig into the lens material and may be more efficient in the sculpt and quadrant technique to break the lens as ultrasonic power is applied as discussed above.

In some examples, tip 124a and tip 124b may be straight or beveled. Straight tips may be rounded and/or polished. The straight tips may also be textured. In some examples, tip 124a and tip 124b may be different shapes. For example, in some examples, the tip 124a may be straight and the tip 124b may beveled.

The length of the tines 121a and 121b may be optimized to improve needle movement while balancing the aspiration flow. For example, shorter tines move at a higher frequency (i.e., more quickly) compared to longer tines. However, shorter tines may have less transverse motion compared to longer tines. In one example, the tines may be between 0.050 and 0.090 inches.

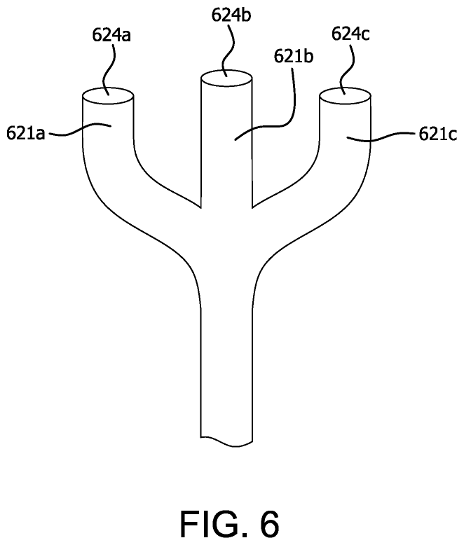

FIG. 1C is a view of a needle 150 of a phacoemulsification handpiece, according to an example. The needle 150 may include a proximal end 160 and a distal end 170. The proximal end 160 may include an elongated needle body 161. The elongated needle body 161 may be screwed on to the distal end of the handpiece using the tool shown in FIGS. 6-8 that couples with at least the tines of the needle 150. The tines 171a and 171b and needle body 161 may be hollow to facilitate aspiration of the lens material and fluid during phacoemulsification.

The elongated needle body 161 may include an outer surface and a central channel or lumen coupled with one or more pumps to aspirate the emulsified lens particles from the eye. The outer surface of the elongated needle body 161 may also include external threads 182 that mate with internal threads of a phacoemulsification handpiece.

In some examples, the distal end 170 of the needle 150 may be a U-shape and may include a first tine 171a and a second tine 171b. The first tine 171a may include a proximal end 172a and a distal end 173a that terminates in a tip 174a. The second tine 171b may include a proximal end 172b and a distal end 173b that terminates in a tip 174b. The first tine 171a and second tine 171b may be connected to the elongated body 161 at a junction point 180. The U-shaped distal end 170 may help dig into the lens material and may be more efficient in the sculpt and quadrant technique to break the lens as ultrasonic power is applied as discussed above.

In some examples, tip 174a and tip 174b may be straight or beveled. Straight tips may be rounded and/or polished. The straight tips may also be textured. In some examples, tip 174a and tip 174b may be different shapes. For example, in some examples, the tip 174a may be straight and the tip 174b may beveled.

The length of the tines 171a and 171b may be optimized to improve needle movement while balancing the aspiration flow. For example, shorter tines move at a higher frequency (i.e., more quickly) compared to longer tines. However, shorter tines may have less transverse motion compared to longer tines. In one example, the tines may be between 0.050 and 0.090 inches.

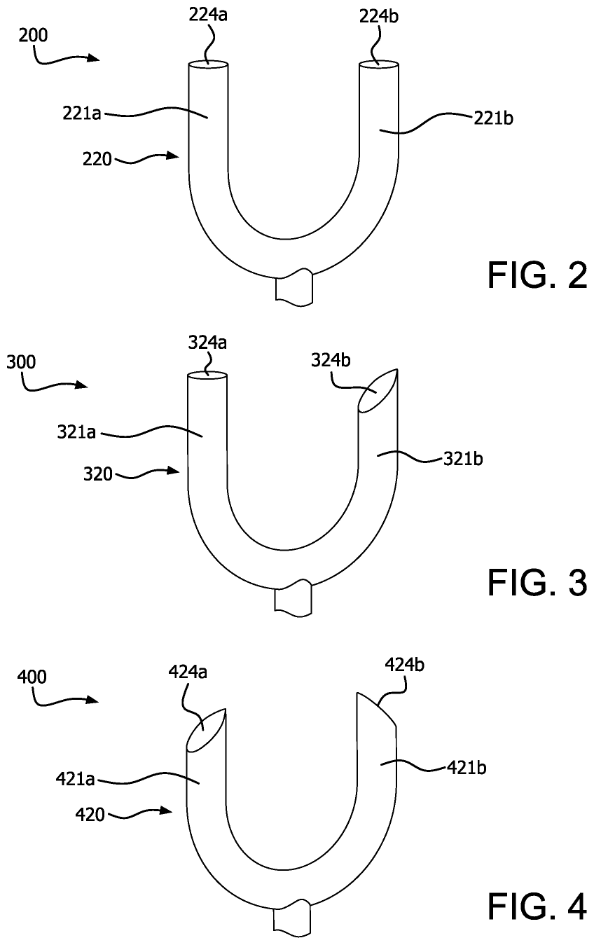

FIG. 2 illustrates an example needle 200 of a phacoemulsification handpiece. Similar to FIG. 1, the needle 200 may include a distal end 220. Also similar to needle 100, the distal end 220 may be a U-shape and may include a first tine 221a and a second tine 221b, where the first tine 221a includes a tip 224a and the second tine 221b includes a tip 224b. As shown in FIG. 2, tip 224a and tip 224b may be straight.

FIG. 3 illustrates an example needle 300 of a phacoemulsification handpiece. Similar to FIG. 1 and FIG. 2, the needle 300 may include a distal end 320. The distal end 320 may be a U-shape and may include a first tine 321a and a second tine 321b, where the first tine 321a includes a tip 324a and the second tine 321b includes a tip 324b. As shown in FIG. 3, the tip 324a may be straight, while the tip 324b may be beveled.

FIG. 4 illustrates an example needle 400 of a phacoemulsification handpiece. Similar to FIG. 1, FIG. 2, and FIG. 3, the needle 400 may include a distal end 420. The distal end 420 may be a U-shape and may include a first tine 421a and a second tine 421b, where the first tine 421a includes a tip 424a and the second tine 421b includes a tip 424b. As shown in FIG. 3, tip 424a and tip 424b may be beveled. The bevel angle may vary from 10 degrees to 50 degrees depending on the preference of the physician. The bevel up (sharp edge) may be made to be up on one tine and down on other or both tines would have bevel up or both may have bevel down. The multi-tine design of this tip provides flexibility for the physician to choose these bevel options.

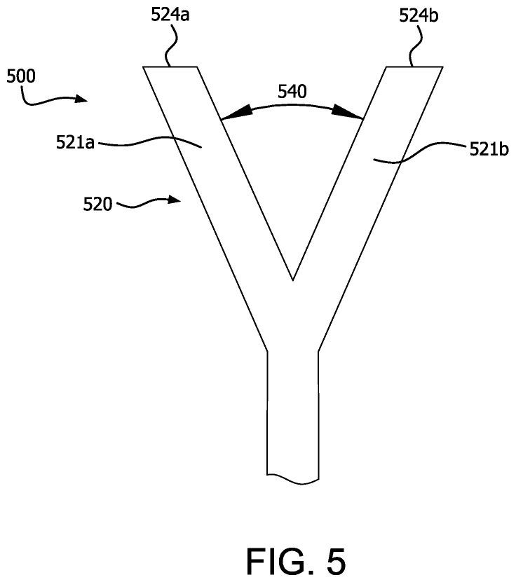

FIG. 5 illustrates an example needle 500 of a phacoemulsification handpiece. Similar to FIG. 1, FIG. 2, FIG. 3, and FIG. 4 the needle 500 may include a distal end 520. However, unlike the previously described needles, the distal end 520 may be a V-shape and may include a first tine 521a and a second tine 521b. The first tine 521a includes a tip 524a and the second tine 521b includes a tip 524b. Tip 524a and tip 524b may be straight or beveled. The angle 540 between tine 521a and tine 521b may be between 10 and 50 degrees.

Although FIGS. 1 through 5 illustrates needles with two tines, it is possible for a needle to include three or more tines. The additional tines may be in the same plane as the first and second tines, but may also be in a different plane such as to make a three-dimensional needle. For example, if the first and second tine are in the y-direction, the additional tine(s) may also be in the y-direction or may be in the z-direction.

FIG. 6 illustrates a needle with three tines. The needle shown in FIG. 6, may include the same features of the needles described in FIGS. 1 to 5 above. As shown in FIG. 6, a three-tine needle may include a first tine 621a, second tine 621b, and a third tine 621c. First tine 621a may include a tip 624a, second tine 621b may include a tip 624b, and the third tine 621c may include a tip 624c. The tines 621a, 621b, and 621c may be hollow to facilitate aspiration of the lens material and fluid during phacoemulsification. In some examples, tip 624a, tip 624b, and tip 624c may be straight or beveled. In some examples, tip 624a, tip 624b, and tip 624c may be different shapes.

FIG. 7a illustrates an example tool 700 to assemble and disassemble the previously discussed needles of a phacoemulsification handpiece. In an example, the example tool may be a comparable to a wrench to assemble and disassemble the needle to the handpiece.

As seen in FIG. 7a, the tool 700 may include two vertical portions 711a and 711b and one horizontal portion 712. The horizontal portion 712 may be longer than the gap 714 between vertical portion 711a and 711b. The tool 700 may resemble the letter “H” where the horizontal portion 712 extends past the vertical portions 711a and 711b. The vertical portions 711a and 711b and horizontal portion 712 may be made of titanium, stainless steel or any material suitable for phacoemulsification.

Each of the vertical portions 711a and 711b may have a hollow portion with a circumference that is substantially similar to the circumference of the tines disclosed in FIGS. 1 to 5 such that tines fit into each of the vertical portions 711a and 711b (i.e., one tine fits into one vertical portion). The inside surface of the vertical portions 711a and 711b may have a coating or an insert with soft feature such as silicone or rubber to grip onto the tines. The tool 700 may be used to assemble and disassemble the U-shaped needles shown in FIGS. 1 to 4.

To install the tip into the handpiece, a user would use the tool 700 and place the vertical portions 711a and 711b over the tines such that the tines are temporarily located within the vertical portions 711a and 711b. The user would then couple the proximal end of the tip with the connection point on the handpiece meant to hold the tip. The user would then turn the tool 700 so as the tighten onto or secure the tip to the connection point. To disassemble the tip, the user would turn the tool 700 in the opposite direction. The horizontal portion 712 may act as a handle such that the user can grip the tool and turn the tines in the appreciate direction.

FIG. 7b illustrates an example tool 750 to assemble and disassemble the previously discussed tips of a phacoemulsification handpiece. As shown, FIG. 7b is similar to FIG. 7a except the vertical portions 761a and 761b do not rise above the horizontal portion. Tool 750 may be used in the same manner as tool 700.

FIG. 8a illustrates an example tool 800 to assemble and disassemble the previously discussed tips of a phacoemulsification handpiece. Similar to tool 700, tool 800 may include two vertical portions 811a and 811b and one horizontal portion 812. In an example, the tool may be comparable to a wrench to assemble and disassemble the tip to the handpiece. Tool 800 may be used to assemble and disassemble the tip 500 shown in FIG. 5.

Tool 800 may also include two inwardly angled vertical portions 813a and 813b. The two flanges 813a and 813b are inwardly angled such that they form a “V-shape.” Similar to tool 700, tool 800 includes a horizontal portion 812 that may be longer than the top gap between portions 813a and 813b, such that it extends past the outer plane of each of the portions 813a and 813b.

Each of the flanges 813a and 813b may have a hollow portion with a circumference that is substantially similar to the circumference of the tines disclosed in FIG. 5 such that tines fit into each of the portions 511a and 511b (i.e., one tine fits into one portion). The inside of the portions 813a and 813b may include some friction to better hold the tines.

The tool 800 may have an openings 830a and 830b to facilitate insertion of the tip. The tines of the tip may be first be inserted from the flange 813a and then pushed up and aligned against flange 813b so the tines of the needle interface with the inner surface of the flange 813a and 813b interfaces such that the tool 800 can apply torque to screw the needle into and out of the phacoemulsification handpiece.

FIG. 8b illustrates an example tool 850 to assemble and disassemble the previously discussed tips of a phacoemulsification handpiece. In an example, the tool may be a comparable to a wrench to assemble and disassemble the tip to the handpiece. Tool 850 may be used to assemble and disassemble the tip 500 shown in FIG. 5.

As shown in FIG. 8b, tool 850 may be similar to tool 800 except that it does not include the two vertical portions 811a and 811b. Similar to tool 800, tool 850 includes a horizonal portion 861, two inwardly angled flanges 863a and 863b, and openings 880a and 880b.

FIG. 9 illustrates the top portion of an example tool 900 to assemble and disassemble the previously discussed tips of a phacoemulsification handpiece. Similar to tool 850, tool 900 may include a horizontal portion 901, two inwardly angled flanges 963a and 963b, and openings 980a and 980b. Each end of the horizonal portion 901 may include indent 920a and indent 920b that may allow a user to better grip the tool as they assemble and/or disassemble the phacoemulsification tip to/from the handpiece.

Although the examples described herein mainly address phacoemulsification, the methods and systems described herein can also be used in other applications that may require a tip.

It will thus be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

What is claimed is:

1. A needle for use with a phacoemulsification handpiece comprising:
an elongated needle body having a proximal end and a distal end terminating at a junction point;
a first tine having a proximal end and a distal end terminating in a first tip;
a second tine having a proximal end and a distal end terminating in a second tip;
wherein the proximal end of the first tine and the proximal end of the second tine are each connected at the junction point.

2. The needle of claim 1, wherein the first tine and the second tine each have an outward curve to form a U-shape.

3. The needle of claim 1, wherein a distance between the first tip and the second tip is larger than the distance between the proximal end of the first tine and the proximal end of the second tine near the junction point to form a V-shape.

4. The needle of claim 1, wherein the first tip is one of straight or beveled.

5. The needle of claim 1, wherein the second tip is one of straight or beveled.

6. The needle of claim 1, wherein the needle body, the first tine, and the second tine include an aspiration lumen therethrough.

7. The needle of claim 1, wherein the needle body includes internal threads.

8. The needle of claim 1, further comprising one or more additional tines.

9. The needle of claim 8, wherein the one or more additional tines are in the same plane as the first tine and the second tine.

10. The needle of claim 8, wherein the one or more additional tines are not in the same plane as the first tine and the second tine.

11. A device for installing a needle in a phacoemulsification handpiece comprising, the device comprising:
a first vertical portion and a second vertical portion, wherein the first vertical portion includes a first hollow portion and the second vertical portion includes a second hollow portion; and
a first horizontal portion connecting the first vertical portion and the second vertical portion;
wherein the first hollow portion and second hollow portion each have a circumference substantially similar to a first tine and a second tine of the phacoemulsification needle and are configured to receive the first tine and the second tine.

12. The device of claim 11, wherein the first vertical portion and the second vertical portion are made of titanium or stainless steel.

13. The device of claim 11, wherein an inside surface of the first hollow portion and the second hollow portion include a highly frictional surface.

14. The device of claim 13, wherein the highly frictional surface includes silicon.

15. The device of claim 13, wherein the highly frictional surface includes rubber.

16. A phacoemulsification needle system comprising:
phacoemulsification needle, comprising:
an elongated needle body having a proximal end and a distal end terminating at a junction point;
a first tine having a proximal end and a distal end terminating in a first tip;
a second tine having a proximal end and a distal end terminating in a second tip;
wherein the proximal end of the first tine and the proximal end of the second tine are each connected at the junction point,
a phacoemulsification needle tool, comprising:
a first vertical portion and a second vertical portion, wherein the first vertical portion includes a first hollow portion and the second vertical portion includes a second hollow portion; and
a first horizontal portion connecting the first vertical portion and the second vertical portion;
wherein the first hollow portion and second hollow portion each have a circumference substantially similar to the first tine and the second tine and are configured to receive the first tine and the second time.
